(19) United States
(12) Patent Application Publication
(10) Pub. No.: US 2026/0266809 A1
(43) Pub. Date: Sep. 10, 2026

(54) MOLECULAR IMAGING AGENTS AND METHODS OF MAKING AND USING THE SAME

(71) Applicant: Vanderbilt University, Nashville, TN (US)

(72) Inventors: Adam Rosenberg, Nashville, TN (US); Eben Rosenthal, Nashville, TN (US); Marisa Hom, Nashville, TN (US); Kamal Jouad, Nashville, TN (US)

(21) Appl. No.: 19/559,520

(22) Filed: Mar. 6, 2026

Related U.S. Application Data
(60) Provisional application No. 63/768,471, filed on Mar. 7, 2025.

(51) Int. Cl. G01N 33/533; B01D 15/34; C07K 16/28

(52) U.S. Cl. CPC G01N 33/533; B01D 15/34; C07K 16/2818; C07K 16/2863; C07K 2317/10

(57) ABSTRACT

Presented herein are optically-labelled therapeutics, such as optically-labelled antibodies, and methods and systems for making the same.

### CROSS REFERENCE TO RELATED APPLICATION

This application claims the benefit of U.S. Provisional Application No. 63/768,471, filed on Mar. 7, 2025, the entire contents of which are incorporated by reference herein.

### FIELD OF THE INVENTION

The present invention relates to optically-labelled therapeutics, such as antibodies, and methods and systems for making the same.

### BRIEF SUMMARY OF THE INVENTION

Monoclonal antibodies have shown great potential in the clinical field of cancer treatment and diagnosis by being conjugated to a cytotoxic drug or to an imaging agent. Nevertheless, their method of bioconjugation is yet traditionally manufactured. For this purpose, we designed an automated optical labeling of monoclonal antibodies, such as panitumumab and nivolumab, based on single-use components utilizing an automated synthesis module. The advantage of utilizing a radiosynthesis module with single-use technologies for optical labeling consists in pre-sterilized components that can be easily replaced between batches, eliminating the need for cleaning and validation. Building upon a recently reported automated method for the clinical production of an ⁸⁹Zr-labelled monoclonal antibody, presented herein are systems and methods for the automated optical labeling of monoclonal antibodies. Methodologies according to various aspects of the disclosure use a single-use cassette that is assembled and connected to a disposable size exclusion chromatography (SEC) column along with a closed system centrifuge tube reactor (FIGS. 1-2).

Various non-limiting aspects of the disclosure can be described as follows.

In some instances, a first aspect of the disclosure can be described as a system for the production of optically-labelled therapeutics, where the system comprises an automated synthesis module, a reaction container fluidically coupled with the automated synthesis module and configured to receive a therapeutic compound, a dye and a reaction buffer, a chromatography column fluidically coupled with the reaction container, and a product container configured to receive a purified optically-labelled therapeutic from the chromatography column. The automated synthesis module is fluidically coupled with a first container housing the therapeutic compound, a second container housing the dye couplable with the therapeutic compound, a third container housing the reaction buffer, and a fourth container housing a formulation buffer.

In some instances, a second aspect of the disclosure can be described as a system according to the first aspect, wherein the reaction container is at least partially contained within an external heat source.

In some instances, a third aspect of the disclosure can be described as a system according to the first or second aspect, wherein the chromatography column is a size exclusion chromatography (SEC) column.

In some instances, a fourth aspect of the disclosure can be described as a system according to any one of the first through third aspects, the therapeutic compound is an antibody, a polysaccharide, an oligonucleotide, a peptide, a protein, a glycoprotein, DNA, RNA, or a small molecule.

In some instances, a fifth aspect of the disclosure can be described as a system according to any one of the first through third aspects, wherein the therapeutic compound is an affibody, a minibody, or a nanobody.

In some instances, a sixth aspect of the disclosure can be described as a system according to any one of the first through third aspects, wherein the therapeutic compound is an antibody.

In some instances, a seventh aspect of the disclosure can be described as a system according to the sixth aspect, wherein antibody is an anti-EGFR monoclonal antibody.

In some instances, an eighth aspect of the disclosure can be described as a method for producing an optically-labelled therapeutic using a system according to any one of the first through third aspects, where the method comprises transmitting a therapeutic compound, a dye and a reaction buffer from the automated synthesis to the reaction container, reacting the therapeutic compound and the dye in the reaction container to form a mixture comprising an optically-labelled therapeutic, passing the mixture through the chromatography column to produce a purified optically-labelled therapeutic, and transmitting the purified optically-labelled therapeutic to the product container.

In some instances, a ninth aspect of the disclosure can be described as a method according to the eighth aspect, wherein the optically-labelled therapeutic is an optically-labelled monoclonal antibody.

In some instances, a tenth aspect of the disclosure can be described as a method according to the eighth aspect, wherein the optically-labelled therapeutic is an optically-labelled anti-EGFR monoclonal antibody.

In some instances, an eleventh aspect of the disclosure can be described as a method according to the eighth aspect, wherein the optically-labelled therapeutic is an optically-labelled panitumumab.

In some instances, an twelfth aspect of the disclosure can be described as a method according to the eighth aspect, wherein the optically-labelled therapeutic is an optically-labelled nivolumab.

In some instances, a thirteenth aspect of the disclosure can be described as a method according to the eighth aspect, wherein the optically-labelled therapeutic is an optically-labelled antibody, polysaccharide, oligonucleotide, peptide, protein, glycoprotein, DNA, RNA, or small molecule.

In some instances, a fourteenth aspect of the disclosure can be described as a method according to the eighth aspect, wherein the optically-labelled therapeutic is an optically-labelled affibody, minibody, or nanobody.

In some instances, a fifteenth aspect of the disclosure can be described as an optically-labelled therapeutic produced by a system according to any one of the first through seventh aspects.

In some instances, a fifteenth aspect of the disclosure can be described as an optically-labelled therapeutic produced by a method according to any one of the eighth through fourteenth aspects.

### BRIEF DESCRIPTION OF THE DRAWINGS

FIG. 1 is a schematic diagram illustrating an exemplary optic labelling module (or system), according to various aspects of the disclosure, comprising a single-use synthesis cassette that is assembled and fluidically coupled with a disposable size exclusion chromatography (SEC) column and a closed system reactor vessel.

FIG. 2 is a photograph of an exemplary optic labelling module (or system), according to various aspects of the disclosure, comprising a single-use synthesis cassette that is assembled and fluidically coupled with a disposable size exclusion chromatography (SEC) column and a closed system reactor vessel.

FIG. 3 provides SEC-HPLC chromatograms of panitumumab at 280 nm and 780 nm, respectively.

FIG. 4 provides SEC-HPLC chromatograms of IRDye® 800CW carboxylate at 280 nm and 780 nm, respectively.

FIG. 5 provides SEC-HPLC chromatograms of IRDye® 800CW NHS-ester at 280 nm and 780 nm, respectively.

FIG. 6 provides SEC-HPLC chromatograms of an optically labelled antibody according to various aspects of the disclosure, panitumumab-IRDye® 800CW, at 280 nm and 780 nm, respectively.

### DETAILED DESCRIPTION

Molecular imaging of targeted therapeutics provides unique opportunities to improve clinical care. Currently, radiolabeling therapeutics is a widely accepted tool to identify disease, measure biological processes, calculate injected dose per gram in organs, and whole body distribution. Recently, optical labeling of cancer targeted agents has been developed for intraoperative molecular imaging (visualizing disease across multiple cancer types) and for high resolution measurements of drug delivery. To date, optical labeling of proteins for administration to humans has not been established and remains cost-prohibitive. Optical agents require the manufacture of medium to large batches at contract research organizations (CROs), which is costly and labor intensive. Costs associated with manufacture for human use have severely restricted their use in molecular imaging in patients. To that end, we present herein a novel manufacturing methodology that could lower the resource requirements and cost to produce labeled molecular imaging agents and allow widespread utilization.

Presented herein are systems and methodologies for producing optically-labelled therapeutics, such as antibodies, by use of established FDA-approved automated synthesis modules that, until now, were adopted for the manufacture of radio-labeled molecular imaging agents. These modules, typically the size of a standard microwave, are operated remotely via a personal computer. For radiochemistry purposes, they are located in “hotcells” which shield the radiation from the users and, if required, in environmentally classified areas. Cassette-based modules utilize a single-use sterilized disposable cassette that contains all of the reagents, reactors, purification media, and flow-paths required for the radiosynthesis. These modules represent an ideal platform to develop molecular imaging agents from clinical grade therapeutics due to their existing GMP-capabilities, small size, and regulatory clearance. We hypothesize that repurposing radiosynthesis modules for use in optical labeling of therapeutics, such as antibodies, will revolutionize molecular imaging.

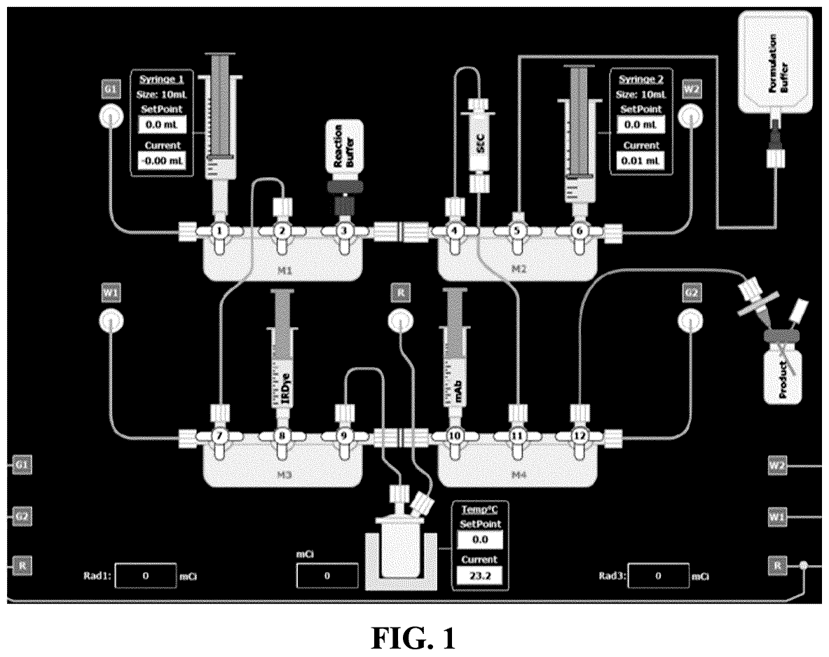

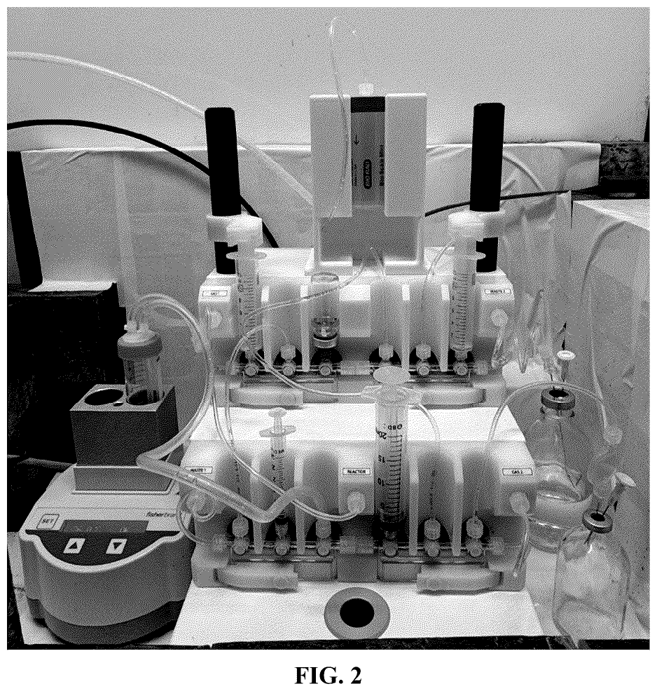

Monoclonal antibodies have shown great potential in the clinical field of cancer treatment and diagnosis by being conjugated to a cytotoxic drug or to an imaging agent. Nevertheless, current methods for their bioconjugation rely upon traditional, bench-top chemistries. To improve upon the synthesis of, we designed an automated optical labeling of monoclonal antibodies, such as panitumumab and nivolumab, based on single-use components utilizing an automated synthesis module. The advantage of utilizing a radiosynthesis module with single-use technologies for optical labeling consists in pre-sterilized components that can be easily replaced between batches, eliminating the need for cleaning and validation. Building upon a recently reported automated method for the clinical production of a ⁸⁹Zr-labelled monoclonal antibody, we have developed an automated optical labeling of monoclonal antibodies. Methodologies for synthesis of optically-labeled monoclonal antibodies according to various aspects of the disclosure are based on the use of a single-use cassette that is assembled and fluidically coupled with a disposable size exclusion chromatography (SEC) column along with a closed system reactor vessel, such as a 50 mL centrifuge tube (FIGS. 1-2). FIG. 1 is a schematic illustration of an exemplary optic labelling module (or system) according to various aspects of the disclosure. FIG. 2 is a photograph of an exemplary optic labelling module (or system) according to various aspects of the disclosure.

Using optic labelling modules (or systems) according to various aspects of the disclosure, Good Manufacturing Practice (GMP)-compliant methodologies for the automated manufacture of optically-labelled molecular imaging agents (or optically-labelled therapeutics) can be realized using various cassette designs with precise control over reagent stoichiometry, reaction temperature, reagent type (reaction buffer, dye coupling partner (NHS-Ester vs. Maleimide), and so one), and purification methods. Such improvements to the synthesis of optically-labelled antibodies will result in increases in chemical yield and quality of the optical imaging agents produced.

Additionally, using optic labelling modules (or systems) according to various aspects of the disclosure, chemistry-based cleanup methods are presented herein to remove unconjugated dye so as to eliminate or reduce the need for extensive purification of the target optically-labelled therapeutic. The use of solid-supported reagents, click-chemistry methods, and development of solid-supported optical dyes in this regard is envisioned.

Furthermore, using optic labelling modules (or systems) according to various aspects of the disclosure, the gram-scale production of optically labeled molecular imaging agents in conformity with GMP standards is envisioned.

Various dyes may be used in systems and methods according to various aspects of the disclosure to couple with a therapeutic compound and form an optically-labelled therapeutic. Exemplary dyes include, but are not limited to, IRDye® 800CW NHS-ester (CAS No. 956579-01-4), succinimidyl ester, sulfo-NHS-LC-biotin, amine-reactive and TFP ester pHrodo dyes, maleimide-PEG2-biotin, 6-carboxamido-(6-azidohexanyl), triethylammonium salt) 6-isomer, NHS ester-activated DyLight Fluors, tetramethylrhodamine (TAMRA) azide, tetramethylrhodamine-5-iodoacetamide dihydroiodide, tetramethylrhodamine-5-maleimide, iodoacetyl-LC-biotin, Alexa Fluor™ 555 Cadaverine, Alexa Fluor™ 488 Cadaverine, Alexa Fluor™ 647 Cadaverine, Alexa Fluor™ 594 Cadaverine, Alexa Fluor™ 594 Hydrazide, Alexa Fluor™ 350 Hydrazide, Alexa Fluor™ 568 Hydrazide, Alexa Fluor™ 633 Hydrazide, Alexa Fluor™ 647 Hydroxylamine, Alexa Fluor™ 488 Hydroxylamine, 5-TAMRA (5-Carboxytetramethylrhodamine, succinimidyl ester), 6-TAMRA (6-Carboxytetramethylrhodamine), L-homopropargylglycine, L-azidohomoalanine, 15-azidopentadecanoic acid, 12-azidododecanoic acid), psoralen-PEG3-biotin, 5-DTAF (5-(4,6-dichlorotriazinyl) aminofluorescein), BODIPY™ FL (4,4-difluoro-5,7-dimethyl-4-bora-3a, 4a-diaza-s-indacene-3-propionic acid), DyLight™ 594 Maleimide, DyLight™ 800 Maleimide, DyLight™ 488 Maleimide, FITC (5/6-fluorescein isothiocyanate), and NHS-Fluorescein (5/6-carboxyfluorescein succinimidyl ester). This list is not exclusive. Generally, any dye that may react with a reactive functional group of a therapeutic compound may be used. Exemplary reactive functional groups include, but are not limited to, amines, amides, ketones, thiols, carboxylates, and hydroxyls.

Systems and methods according to various aspects of the disclosure may be used to couple dyes with various types of therapeutic compounds to form optically-labelled therapeutics. Exemplary therapeutic compounds include, but are not limited to, antibodies polysaccharides, oligonucleotides, peptides, proteins, glycoproteins, DNA, RNA, small molecules, affibodies, minibodies, nanobodies.

### EXAMPLES

Materials. In the Examples below, the following reagents and buffers were used as received without further purification: Panitumumab (300 mg, Amgen, NDC 55513-954-01), 1.0 M Potassium Phosphate Dibasic aqueous solution (6 mL, Millipore-Sigma), 2×250 mL bottles of 50 mM Sodium Phosphate with 2% mannitol, pH-7.4 (Teknova), IRDye® 800CW NHS-ester (15 mg, LI-COR, CAS No. 956579-01-4), Cell culture grade water (1 mL, Corning, 25-055-CI), ultrapure water (1 L, made from a Milli-Q® water purification system), Sodium azide (0.65 g, Sigma Aldrich, 26628-22-8), 10× Phosphate Buffered Saline (5 mL, Cytiva, SH30258.01), and 5 M sodium chloride solution (30 mL, Corning, 46-032-CV).

In the Examples below, the following equipment were used: MultiSyn radiosynthesizer cassette (iPhase technologies Pty Ltd.; also referred to herein as a radiosynthesis cassette or cassette) with MultiSyn software package, external heating block (Fisher Scientific, 14-955-241), HPLC system equipped with a 280 nm and 780 nm channels detector, HPLC column: Superdex™ 200 Increase 10/300GL (Cytiva, 28-9909-44, column L×I.D. 30 cm×10 mm, 8.6 μm particle analytical column), Bio-Scale™ Mini Bio-Gel® P-6 desalting cartridge 50 mL (size exclusion chromatography (SEC) column, Bio-Rad, 7325312), NanoDrop™ One UV/Vis system (Thermo Scientific), spectrophotometer UV/Vis with quartz cuvette to scan range of 200-800 nm, sterile disposable MultiSyn cassette MSH-310 Optical mAb Radiolabeling (iPhase technologies Pty Ltd.), Sterile 50 mL closed system centrifuge tube-self-standing with dip tube (product reactor vessel, Corning, 11705), Luer lock sterile 2-part NORM-JECT® syringes, 10 mL (Henke Sass Wolf, 8300027527), Luer lock sterile 3-part syringe, 3 mL, 20G×1″ (Becton-Dickinson, 309579), Luer lock sterile 3-part syringes 20 mL (Becton-Dickinson 302030), 18G×6″ spinal needles (Medline PAIN8033), 21G×2″ sterile needles (Becton-Dickinson), 18G×1½″ sterile needles (Becton-Dickinson, 305196), Sterile filtered vent needles (International Medical Industries 31-20), 0.22 μm Pall filters (Pall AEF1NTE), 100 mL sealed sterile vial (ALK SEV2072022), 10 mL sealed sterile vials (ALK SEV1020), sterile septum caps (ThermoFisher Scientific 02-923-20), glass vacuum filtration distillation apparatus (2000 mL; includes 1 L graduated funnel filter flask, clamp 10-micron pore size compatible with 42-60 mm membrane filter paper and 0.45-micron membrane filter paper), tryptic soy agar (Settling Plate, Remel, R01920), contact plate 1 TSA w/polysorbate 80 (Remel R111101), and tube sealed blood bags.

Preparation of Optic Labelling System (or Module). The optic labelling system was prepared for use prior to experimentation using the following procedure. First, air and nitrogen were introduced into the system to achieve a nitrogen pressure of >500 psi with the regulator set to 15.0 to 20.0 psi. The system is maintained between reaction runs by cleaning or replacing one or system components including syringes, buffer vials/flasks, the chromatography purification (SEC) column, sterile product reactor vessel. Finally the isotherm dry bath is turned on and the temperature is set to 25° C.

Preparation of cassette assembly. Synthesis cassettes for use in the optic labelling system are assembled using the following procedure. First, the cassette manifold and stopcock manifolds are visually inspected, ensuring they are not cracked or otherwise compromised. Next, the rotating taps are oriented away from the user and it is verified that the taps are oriented for proper installation of the cassette within the module. Then, the 18.5 cm tube is removed from position 9 of the module (see FIG. 1) and connected to the 50 mL reactor filter port. Finally, the 10 mL two-piece syringes are placed on the cassette at position 1 and 6 (see FIG. 1), ensuring a good connection.

Cassette installation into system. The cassette is installed optic labelling module using the following procedure. First, the top manifolds of the cassette are installed and the syringes are secured to the plungers at positions 1 and 6 (see FIGS. 1 and 2). Next, the bottom manifolds of the cassette is installed. Then, the reactor and a central reactor gas/vacuum connection are connected to each other with a tube. Next, the waste and gas connections of manifold positions 1, 2, 3 and 4 are connected to the system. All magnetic clamp arms are then closed to secure the cassette in the system. Finally, the reactor (sterile product reactor vessel) is placed in the 25° C. isotherm dry bath.

Further assembly of system. After installation of the cassette, the following steps are performed. First, the preassembled IPM Sterile 100 mL filtered bulk vial on the Pall filter is connected to position 12 of the cassette via tubing.

Next, the SEC column is connected to positions 4 and 11 of the cassette via tubing.

Then, a reaction buffer vial (here, a 10 mL sterile vial containing 1 M potassium phosphate buffer) is connected to position 3 of the cassette via tubing, a formulation buffer container (here, a 20 mL bottle containing 50 mM Sodium Phosphate with 2% Mannitol, pH 7.4) is connected to position 5 of the cassette via tubing, a monoclonal antibody (mAb) container (here, a Panitumumab solution) is connected to position 10 of the cassette via tubing, and a dye container (here, a syringe containing a dye solution of IRDye® 800CW NHS-ester in cell culture water) is connected to position 8 of the cassette via tubing.

Upon completion of the above, the optic labelling system will appear as shown in FIG. 2.

Automated Synthesis of Optically-Labelled Monoclonal Antibodies (mAbs). Optically-labelled mAbs are prepared by conjugating an mAb and an optically active label using the optic labelling system with a process sequence generated using the MultiSyn software package. The process sequence provides for automated addition of the reagents, followed by equilibration of the size exclusion chromatography, reaction mixture homogenizing, purification and collection of the final product. Upon initiation of the process sequence, the following steps are performed. First, the reactor (sterile product reactor vessel) is placed under vacuum and the dye solution is drawn from its syringe and transferred to the reactor. Upon completion of the transfer, the reactor is depressurized with a low flow of nitrogen.

Next, the reaction buffer vial is pressurized with a low flow of nitrogen. Using the syringe at position 1, a first portion of the reaction buffer is transferred to the waste and a second portion is transferred to the reactor. For example, in this step, the first portion is 2.0 mL and the second portion an amount sufficient to adjust the pH of the reaction mixture in the reactor to pH=8.2±0.3 for the conjugation (i.e., optical labelling of the mAbs).

Then, the reactor is again placed under vacuum and a portion of mAb solution is transferred from the mAb container to the reactor. For example, in this step, the portion of mAb solution is a 15 mL solution having 300 mg of panitumumab.

Using the syringe at position 6, the SEC column is conditioned using the formulation buffer during the conjugation reaction. The lines between position 1 to position 6 and position 1 to position 7 should be dried using a dual nitrogen/vacuum flow after the SEC cartridge has been equilibrated. This prevents the formulation buffer, which contains sugars, from being transferred to the reactor while the reaction mixture is bubbling. Consequently, absence of formulation buffer in the reactor prevents undesired susceptible trans-esterification with the IRDye® 800CW NHS ester which may cause a low degree of labelling.

The conjugation reaction is conducted for a predetermined temperature and period of time (for example, 25° C. for 1 hour). To homogenize the solution in the reactor, the reaction mixture can be intermittently bubbled with nitrogen (for example, for 30 seconds after every 10 minutes during the reaction time period). Once the conjugation reaction is completed, the crude product is purified (by, for example, size exclusion chromatography) to eliminate the excess free dye. Specifically, the crude product is loaded on the SEC column using the syringe at position 1. The loading step includes one or more rinses of the syringe at position 1 and the lines with formulation buffer. This rinse(s) are then loaded onto the SEC column and collected in the final product vial at position 12. The conjugate trapped in the SEC column is then eluted with formulation buffer using the syringe at position 6. The final purified optically-labelled mAb is then collected in the final product vial at position 12 and stored in a light blocking material (e.g., aluminum foil) and under refrigeration (2-8° C.).

Results. Currently, fluorescently labelled anti-EGFR monoclonal antibodies, such as panitumumab² and cetuximab³, are manually produced using 2.3 dye equivalent of IRDye® 800CW NHS-ester under soft conditions, such as stirring at room temperature for two hours with a slightly basic buffer K₂CO₃ or K₂HPO₄ with a pH of 8.5. With these described conditions in hand, we performed an automated production as presented in Table 1, Entry 1. We dissolved 2.8 equivalent of the dye in the reaction buffer (1.0 M K₂HPO₄) at a concentration of 15 mg/mL. To this solution, panitumumab was added followed by the reaction buffer (0.1 mL of 1.0 M K₂HPO₄/1 mL of panitumumab). After 2 h of reaction with a nitrogen stream flow every 10 min, the reaction mixture was loaded and passed through a P-6 SEC column. The panitumumab-IRDye® 800CW was isolated with 0.79 D/P ratio.

TABLE 1

| | | | IRDye800 NHS ester | | | | | | |
|---|---|---|---|---|---|---|---|---|---|
| | | Order of | | | IRDye | Rxn | D/P ratio | | |
| | | reagent | IRDye | IRDye | Conc. | mixture | 15 | 60 | 120 |
| Entry | Labelling | addition | Solvent | Equivalents | (mg/mL) | pH | min | min | min |
| 1 | Automated | IRDye, 1M K₂HPO₄, | 1M K₂HPO₄ | 2.88 | 15 | ND | ND | ND | 0.79 |
| | | panitumumab | | | | | | | |
| 2 | Automated | IRDye, 1M K₂HPO₄, | 1M K₂HPO₄ | 2.88 | 15 | 8.8 | 0.8 | 0.8 | 0.8 |
| | | panitumumab | | | | | | | |
| 3 | Manual | IRDye, 1M K₂HPO₄, | H₂O | 2.88 | 15 | 8.12 | 1.28 | 1.28 | 1.28 |
| | | panitumumab | | | | | | | |
| 4 | Manual | Panitumumab, 1M | H₂O | 2.88 | 15 | 8.13 | 1.23 | 1.23 | 1.23 |
| | | Na₂HPO₄, IRDye | | | | | | | |
| 5 | Manual | Panitumumab, 1M | H₂O | 2.88 | 15 | 8.01 | 1.34 | 1.34 | 1.34 |
| | | Na₂HPO₄, IRDye | | | | | | | |
| 6 | Manual | Panitumumab, 1M | H₂O | 2.88 | 15 | 8.04 | 1.66 | 1.66 | 1.66 |
| | | Na₂HPO₄, IRDye* | | | | | | | |
| 7 | Manual | Panitumumab, 1M | H₂O | 5.6 | 15 | 7.9 | 2.87 | 2.91 | 2.93 |
| | | Na₂HPO₄, IRDye | | | | | | | |
| 8 | Manual | Panitumumab, 1M | H₂O | 5.6 | 5 | 7.95 | 2.7 | 2.7 | 2.71 |
| | | Na₂HPO₄, IRDye | | | | | | | |
| 9 | Automated | IRDye, 1M K₂HPO₄, | H₂O | 5.6 | 5 | 8.83 | ND | 2.62 | ND |
| | | Panitumumab | | | | | | | |
| 10 | Automated | IRDye, 1M K₂HPO₄, | H₂O | 5.6 | 5 | ND | ND | 2.35 | ND |
| | | Panitumumab | | | | | | | |

(ND: not determined; *5 mg of the NHS-ester IRDye800CW dissolved as received then partitioned without prior weighting).

Given that the optimal D/P ratio needed for clinical use of panitumumab-IRDye® 800CW to ensure appropriate half-life, comparable binding affinity to the unlabeled panitumumab and achieve a high image signal with a good tumor-to-background ratio, is approximately 2.0±1.0. The Entry 1 result does not meet the release criteria. For this purpose, multiple optimizations has been performed to determine the optimal D/P ratio. we hypothesized that the automated conjugation reaction required longer time than the manual one. We performed a monitoring of the conjugation reaction by HPLC to determine the optimal automated reaction time. According to the monitoring results shown in Table 1, Entry 2, the D/P ratio is 0.8 for 15 min and remains constant for 2 hours. As a result, the conjugation reaction is completed in 15 minutes, which is faster than what has been reported in the literature^{2,3}.

Estimate of the Degree of Labelling (DOL). The degree of labelling (DOL) also known as Dye-to-Protein ratio (D/P) represents the average number of dye molecules that are conjugated to the antibody. Prior optimization studies have revealed issues concerning both under-labeling and over-labeling of antibodies.⁶ Under-labeling does not provide sufficient image signal for effective tumor visualization and diagnosis. On the other hand, over-labeling with fluorophores can alter antigen binding characteristics, reduce half-life, decrease fluorescence via quenching mechanisms and lower the hydrophilic property of the monoclonal antibody which may produce dimmers and aggregates. However, to overcome the issues associated to under- and over-labeling, the optimal D/P ratio of anti-EGFR antibody falls within the range of 1 to 3 dyes per antibody molecule^{2,3,5,7}. This photophysical property intrinsic to the purified conjugate Panitumumab-IRDye® 800CW has been determined using two different methods.

DOL by spectroscopy-UV measurements. The first method, as we described previously², is based on spectroscopy-UV measurement of the maximum absorption intensity of the panitumumab and the IRDye® 800CW at 280 and 780 nm, respectively. The DOL is measured by solving the equation 1:

$$DOL = ( {A}_{780} / {ε}_{dye} ) / [ ( {A}_{280} - 0 \text{.03} \times {A}_{780} ) / {ε}_{Panitumumab} ] \qquad ( Eq . 1 )$$

The molar extinction coefficients of panitumumab (ε_{panitumumab}) and IRDye® 800CW (ε_{dye}) in 1:1 methanol and PBS are 200,000 and 270,000 M⁻¹ cm⁻¹, respectively.

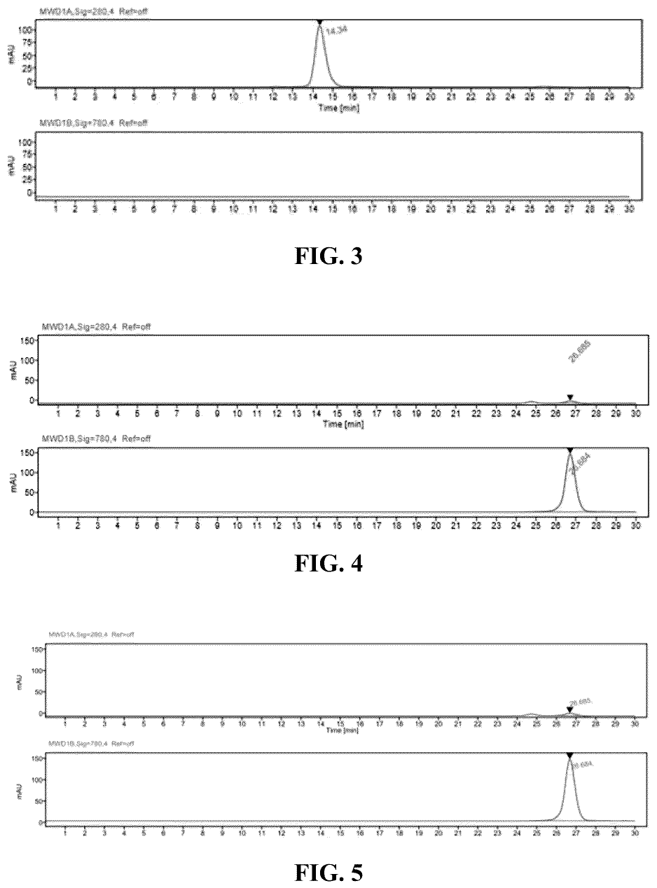

DOL by SEC-HPLC standard calibration curve measurements. The second method involves the use of standard calibration curves by SEC-HPLC to determine the concentrations of the panitumumab and the IRDye® 800CW carboxylate. The standard calibration curves were made by injecting into HPLC triplicate of both standards at different concentrations and plotting the peak area at 14.3 min and 26.6 min against the concentration in mg/mL for panitumumab (FIG. 3) and in ng/μL for IRDye® 800CW carboxylate (FIG. 4) and IRDye® 800CW NHS-ester (FIG. 5) at 280 nm and 780 nm, respectively. Using the standard calibration curves, determine the slope m and m′ of panitumumab and IRDye® 800CW respectively. For each concentration of the dye calibration curve, calculate the % contribution of the IRDye® 800CW carboxylate at 280 nm by dividing the peak area average at 26.6 min at 280 nm to the peak area average at 26.6 min at 780 nm. The average of the resulting % contribution of the different concentrations of the IRDye® 800CW carboxylate at 280 nm will be used in the equation 2.

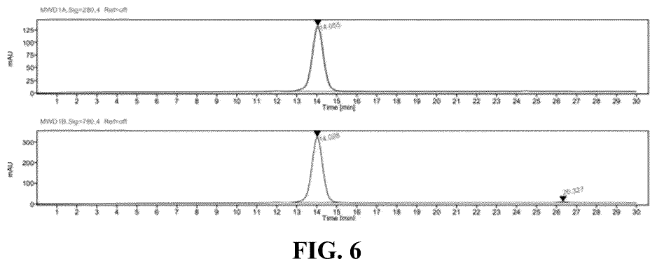

The purified conjugate Panitumumab-IRDye® 800CW was injected into HPLC and the peaks area at 14.3 min and 26.6 min of the signals at 280 and 780 nm (FIG. 6) were measured. The concentration of the panitumumab was obtained by solving the equation 2 and the IRDye® 800CW was directly extrapolated from its calibration curve using equation 3.

The resulting concentrations were divided by the molecular weight of both reagents 145000 and 1091.1 g/mol and converted to mol/L, then used to calculate the estimate the DOL by solving the equation 4.

$$Corrected concentration = \frac{\begin{matrix}Peak area @ 280 nm ( 1 - \% \\ contribution of IRDye 800 @ 280 nm )\end{matrix}}{slope ( m )} ( \frac{mg}{mL} ) \qquad ( Eq . 2 )$$

$$Concentration = \frac{Peak area @ 780 nm}{slope ( {m}^{′} )} ( \frac{ng}{μL} ) \qquad ( Eq . 3 )$$

$$D / P ratio = \frac{Concentration of IRDye 800 CW in mol / L at 780 nm}{\begin{matrix}Corrected concentration of \\ Panitumumab in mol / L at 280 nm\end{matrix}} \qquad ( Eq . 4 )$$

Using the two methods above to quantitatively evaluate the DOL of the panitumumab-IRDye® 800CW conjugate, we found that the first method, based on intensity ratio UV-measurement, gives a D/P ratio value of 1.8 times more than that obtained by the peak area using SEC-HPLC calibration curve method. This result is similarly consistent previous studies which demonstrate that the precision of area ratio is √2 times better than that of intensity ratio. Additionally, in this study we succeeded to monitor, characterize the DOL and optimize the conjugation reaction. These characterizations were achieved without the need to purify the desired product, a process that would not have been feasible using intensity ratio spectroscopy-UV measurements due to the presence of excess free dye in the reaction mixture. Therefore, to circumvent the cost of the single-use SEC purification cartridge and eliminate the need of the purification, we adapted the peak area calibration curve method to optimize and characterize the final drug product production.

The panitumumab-IRDye® 800CW conjugate exhibits the following properties:
Visual appearance: dark green free of visible particles.
pH: 7.4±0.5
Chemical purity: SEC-HPLC (280 and 780 nm), ≥90%
Unbound IRDye® 800CW: SEC-HPLC at 780 nm, ≤5%
Residual NHS: HPLC at 280 nm, ≤0.5%
Degre of Labeling: 1 to 3.
Concentration: 5±0.5 mg/mL
Yield of the conjugation reaction: >90%
Bacterial endotoxin level: ≤5 EU/mL
Sterility test (USP <71>, 14 days)): no growth.

Chemical identity: SDS-PAGE can be conducted under both reducing and non-reducing conditions to estimate the molecular weight and determine the densitometry of bands that constitute more than 5% relative to the unlabeled standard.

Enzyme-linked immunosorbent assay (ELISA) can be conducted to determine the targeting affinity of the fluorescently labelled antibody using an EGF-receptor binding assay.

Synthesis of a Nivolumab-IRDye® 800CW Conjugate. The automated synthesis, using an optic labelling system as described herein, is designed to produce optically-labelled monoclonal antibodies without the need for any pretreatment process including pre-purification and/or concentration. Using the methodology described above for the synthesis of the panitumumab-IRDye® 800CW conjugate, a small scale synthesis of Nivolumab-IRDye® 800CW conjugate was also achieved. Table 2 demonstrates that the conjugation of Nivolumab with the IRDye® 800CW NHS ester resulted in the fluorescently labelled anti-PD-1 product with excellent yield, optimal D/P ratio and high purity.

TABLE 2

| | |
|---|---|
| Initial mass (mg) | 40 |
| Mass collected (mg) | 38.74 |
| Concentration (mg/mL) | 1.49 |
| Yield (%) | 96 |
| Purity (%) @280 nm | 97.3 |
| Purity (%) @780 nm | 99.8 |
| IRDye ® 800CW (%) @780 nm | 0 |
| D/P ratio | 1.65 |
| pH | 7.4 |
| HMWS (%) @280 nm | 0.3 |
| LMWS (%) @280 nm | 2.4 |

In addition to the production of panitumumab-IRDye® 800CW and nivolumab-IRDye® 800CW, the systems and processes disclosed herein have the potential to be applied for the optical labeling (using any suitable optically observable dyes) of a variety of FDA-approved monoclonal antibody formulations that do not contain interfering excipients such as free amino-acids (e.g., L-histidine and L-arginine). For example, in addition to panitumumab and nivolumab, optical labelling of other monoclonal antibodies using methods and systems according to the disclosure may include, for example, cetuximab, bevacizumab, brentuximab vedotin, rituximab, ipilimumab and others. While the use of IRDye® 800CW NHS-ester is highlighted herein as the dye for optic labelling of therapeutics, a wide variety of dyes may be used so long as the dye comprises a functional group capable of binding to a therapeutic to be optically labelled and the dye, upon conjugation, does not interfere with the binding activity of the therapeutic with the therapeutic's target binding site such that the therapeutic no longer functions effectively in its therapeutic use. In general, dyes comprising an activated ester or NHS-ester are suitable for use in accordance with various aspects of the disclosure

Furthermore, the systems and processes disclosed herein may be applied to therapeutics other than antibodies, in instances where, for example, the resulting optically-labelled therapeutics requires preparation in compliance with GMP standards and/or where the optic label does not interfere with the binding activity of the therapeutic with the therapeutic's target binding site such that the therapeutic no longer functions effectively in its therapeutic use. Therapeutics that may be optically labelled using methods and systems according to the disclosure may include, for example, small molecules, peptides, affibodies, minibodies, nanobodies, and so on.

The following references may be pertinent to the present application.
Wichmann, C. W. et al. Automated radiosynthesis of [89Zr]Zr-DFOSq-Durvalumab for imaging of PD-L1 expressing tumours in vivo. Nuclear Medicine and Biology 120-121, 108351 (2023).
Bhattacharyya, S. et al. Synthesis and biological evaluation of panitumumab-IRDye800 conjugate as a fluorescence imaging probe for EGFR-expressing cancers. MedChemComm 5, 1337-1346 (2014).
Zinn, K. R. et al. IND-Directed Safety and Biodistribution Study of Intravenously Injected Cetuximab-IRDye800 in Cynomolgus Macaques. Molecular Imaging and Biology 17, 49-57 (2015).
Heuveling, D. A. et al. Nanocolloidal albumin-IRDye 800CW: a near-infrared fluorescent tracer with optimal retention in the sentinel lymph node. Eur J Nucl Med Mol Imaging 39, 1161-8 (2012).
Ter Weele, E. J. et al. Development, preclinical safety, formulation, and stability of clinical grade bevacizumab-800CW, a new near infrared fluorescent imaging agent for first in human use. European Journal of Pharmaceutics and Biopharmaceutics 104, 226-234 (2016).
Vira, S., Mekhedov, E., Humphrey, G. & Blank, P. S. Fluorescent-labeled antibodies: Balancing functionality and degree of labeling. Analytical Biochemistry 402, 146-150 (2010).
Linssen, M. D. et al. Roadmap for the development and clinical translation of optical tracers cetuximab-800CW and trastuzumab-800CW. Journal of Nuclear Medicine 60, 418-423 (2019).
Hagiwara, Y. & Kuwatani, T. Precision comparison of intensity ratios and area ratios in spectral analysis. Scientific reports 14, 22898-22898 (2024).

The description of the present embodiments of the invention has been presented for purposes of illustration but is not intended to be exhaustive or to limit the invention to the form disclosed. Many modifications and variations will be apparent to those of ordinary skill in the art. As such, while the present invention has been disclosed in connection with an embodiment thereof, it should be understood that other embodiments may fall within the spirit and scope of the invention. All patents and publications cited herein are incorporated by reference in their entirety.

What is claimed is:

1. A system for the production of optically-labelled therapeutics, the system comprising:
an automated synthesis module fluidically coupled with:
a first container housing a therapeutic compound;
a second container housing a dye couplable with the therapeutic compound;
a third container housing a reaction buffer; and
a fourth container housing a formulation buffer;
a reaction container fluidically coupled with the automated synthesis module and configured to receive the therapeutic compound, the dye and the reaction buffer;
a chromatography column fluidically coupled with the reaction container; and
a product container configured to receive a purified optically-labelled therapeutic from the chromatography column.

2. The system of claim 1, wherein the reaction container is at least partially contained within an external heat source.

3. The system of claim 1, wherein the chromatography column is a size exclusion chromatography (SEC) column.

4. The system of claim 1, wherein the therapeutic compound is an antibody, a polysaccharide, an oligonucleotide, a peptide, a protein, a glycoprotein, DNA, RNA, or a small molecule.

5. The system of claim 1, wherein the therapeutic compound is an affibody, a minibody, or a nanobody.

6. The system of claim 1, wherein the therapeutic compound is an antibody.

7. The system of claim 6, wherein antibody is an anti-EGFR monoclonal antibody.

8. A method for producing an optically-labelled therapeutic using a system according to claim 1, comprising:
transmitting a therapeutic compound, a dye and a reaction buffer from the automated synthesis to the reaction container;
reacting the therapeutic compound and the dye in the reaction container to form a mixture comprising an optically-labelled therapeutic;
passing the mixture through the chromatography column to produce a purified optically-labelled therapeutic; and
transmitting the purified optically-labelled therapeutic to the product container.

9. The method of claim 8, wherein the optically-labelled therapeutic is an optically-labelled monoclonal antibody.

10. The method of claim 8, wherein the optically-labelled therapeutic is an optically-labelled anti-EGFR monoclonal antibody.

11. The method of claim 8, wherein the optically-labelled therapeutic is an optically-labelled panitumumab.

12. The method of claim 8, wherein the optically-labelled therapeutic is an optically-labelled nivolumab.

13. The method of claim 8, wherein the optically-labelled therapeutic is an optically-labelled antibody, polysaccharide, oligonucleotide, peptide, protein, glycoprotein, DNA, RNA, or small molecule.

14. The method of claim 8, wherein the optically-labelled therapeutic is an optically-labelled affibody, minibody, or nanobody.
